(19) United States
(12) Patent Application Publication
(10) Pub. No.: US 2007/0032557 A1
(43) Pub. Date: Feb. 8, 2007

(54) Method for administering levosimendan

(76) Inventors: Matti Kivikko, Espoo (FI); Robert Padley, Lake Bluff, IL (US); Roopal Thakkar, Grayslake, IL (US); Leticia Delgado-Herrera, Lake Forest, IL (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/355,897

(22) Filed: Feb. 17, 2006

Related U.S. Application Data
(60) Provisional application No. 60/654,087, filed on Feb. 18, 2005.

(51) Int. Cl. A61K 31/122

(52) U.S. Cl. 514/682

(57) ABSTRACT

The invention relates to intermittent administration of an inotropic agent (such as, but not limited to a levosimendan compound) or a pharmaceutically acceptable salt thereof in the treatment of cardiovascular disorders, such as, pulmonary hypertension, myocardial ischemia, acute heart failure, chronic heart failure or acute on chronic heart failure.

This application claims the benefit of priority to U.S. Provisional Application No. 60/654,087, filed on Feb. 18, 2005.

### TECHNICAL FIELD

The present invention relates to a method for the treatment of cardiovascular disorders such as, but not limited to, acute heart failure, chronic heart failure, pulmonary hypertension or myocardial ischemia by intermittent administration of a levosimendan compound or a pharmaceutically acceptable salt thereof, to a patient.

### BACKGROUND OF THE INVENTION

Levosimendan, which is the (−)-enantiomer of [[4-(1,4,5,6-tetrahydro-4-methyl-6-oxo-3-pyridazinyl)phenyl]hydrazono]propanedinitrile, is an inotropic drug substance that is currently used for the short term treatment of patients who suffer from acutely decompensated severe heart failure. The drug increases contractile force of the heart myocardium by enhancing the sensitivity of myofilaments to calcium. Levosimendan is also known to have vasodilatory and phosphodiesterase-inhibitory properties. Levosimendan is administered by intravenous infusion over the period of 24 hours.

Levosimendan and a method for its preparation are described in U.S. Pat. No. 5,569,657. Use of levosimendan in the treatment of myocardial ischemia is described in U.S. Pat. No. 5,512,572. Use of levosimendan in the treatment of pulmonary hypertension is described in U.S. Pat. No. 6,462,045.

Chronic heart failure is typically treated with various drugs including diuretics, digitalis, beta-blockers, ACE inhibitors, angiotensin II blockers and aldosterone inhibitors. However, patients with acutely decompensated heart failure require parenteral inotropic support to reverse the severe loss of myocardial function and contractility. Such continuous intravenous inotropic support is usually a short-term treatment necessary to stabilize the patients and bring them out of a period of decompensation. Prolonged continuous infusion may be necessary in some patients including those waiting for cardiac transplantation. Prolonged continuous inotropic infusions may, however, be associated with drawbacks such as tolerance, tachyphylaxis, weaning problems, increased risk of infections, long-term hospitalization and increased mortality.

### SUMMARY OF THE INVENTION

In one embodiment, the present invention relates to a method of administering a levosimendan compound or a pharmaceutically acceptable salt thereof to a patient. The method comprises the step of administering intermittently a dose of at least 0.0006 mg/kg of a levosimendan compound or a pharmaceutically acceptable salt thereof to a patient. The period between each intermittent dose is at least one hour. More specifically, the period between each intermittent dose can be at least 2 hours, at least 4 hours, at least 6 hours, at least 12 hours, at least 24 hours, at least 30 hours, at least 2 days, at least 3 days, at least 4 days, at least 5 days, at least 6 days, at least 7 days, at least 8 days, at least 12 days, at least 13 days, at least 14 days, at least 21 days, at least 22 days, at least 25 days, at least 28 days, at least 30 days or at least 35 days. For example, a patient can be administered intermittently a dose of 0.04 mg/kg of a levosimendan compound or a pharmaceutically salt thereof wherein the period between each intermittent dose is from 6 to 30 days.

The period between each intermittent dose can be determined by a variety of methods, such as, but not limited to, by echocardiography or by measuring the plasma level of natriuretic peptide.

The patient treated according to the above-described method may be suffering from pulmonary hypertension, myocardial ischemia chronic heart failure, acute heart failure or acute on chronic heart failure.

In another embodiment, the present invention relates to a method of administering a levosimendan compound or a pharmaceutically acceptable salt thereof to a patient suffering from chronic heart failure. The method comprises the step of administering intermittently a dose of at least 0.0006 mg/kg of a levosimendan compound or a pharmaceutically acceptable salt thereof to a patient. The period between each intermittent dose is at least one hour. More specifically, the period between each intermittent dose can be at least 2 hours, at least 4 hours, at least 6 hours, at least 12 hours, at least 24 hours, at least 30 hours, at least 2 days, at least 3 days, at least 4 days, at least 5 days, at least 6 days, at least 7 days, at least 8 days, at least 12 days, at least 13 days, at least 14 days, at least 21 days, at least 22 days, at least 25 days, at least 28 days, at least 30 days or at least 35 days.

In yet another embodiment, the present invention relates to a method of administering an inotropic agent or a pharmaceutically acceptable salt thereof to a patient. The method comprises the steps of: administering a first dose of said inotropic agent or a pharmaceutically acceptable salt thereof other than dobutamine to a patient, followed by a rest period of at least one hour during which time said inotropic agent or a pharmaceutically acceptable salt thereof is not administered and then administering at least a second dose of an inotropic agent or a pharmaceutically acceptable salt thereof. Each dose in said method delivers at least 0.0006 mg/kg of said inotropic agent or a pharmaceutically acceptable salt thereof and at least one inotropic agent used in the dose is a levosimendan compound or a pharmaceutically acceptable salt thereof. More specifically, the period between each dose can be at least 2 hours, at least 4 hours, at least 6 hours, at least 12 hours, at least 24 hours, at least 30 hours, at least 2 days, at least 3 days, at least 4 days, at least 5 days, at least 6 days, at least 7 days, at least 8 days, at least 12 days, at least 13 days, at least 14 days, at least 21 days, at least 22 days, at least 25 days, at least 28 days, at least 30 days or at least 35 days. For example, a patient can administered intermittently a dose of 0.04 mg/kg of a levosimendan compound or a pharmaceutically salt thereof wherein the period between each intermittent dose is from 6 to 30 days.

The period between each intermittent dose can be determined by a variety of methods, such as, but not limited to, by echocardiography or by measuring the plasma level of natriuretic peptide.

The patient treated according to the above-described method may be suffering from pulmonary hypertension, myocardial ischemia, chronic heart failure, acute heart failure or acute on chronic heart failure.

In yet another embodiment, the present invention relates to a method of administering a levosimendan compound or a pharmaceutically acceptable salt thereof to a patient. The method comprises the step of administering to a patient intermittent intravenous infusions, each infusion delivering a dose of more than 0.0006 mg/kg of a levosimendan compound or a pharmaceutically acceptable salt thereof to a patient, and wherein the period between each intermittent infusion is at least 1 hour.

The period between each intermittent dose can be at least 2 hours, at least 4 hours, at least 6 hours, at least 12 hours, at least 24 hours, at least 30 hours, at least 2 days, at least 3 days, at least 4 days, at least 5 days, at least 6 days, at least 7 days, at least 8 days, at least 12 days, at least 13 days, at least 14 days, at least 21 days, at least 22 days, at least 25 days, at least 28 days, at least 30 days or at least 35 days. For example, the administration of each intermittent intravenous infusion can be over the period of 1 hour to 2 days, from 4 to 36 hours, from 6 to 30 hours, for less than 24 hours, for 7 to 22 days, for 7 to 16 days or for 8 to 13 days.

By way of another example, a patient can be administered intermittently a dose of 0.04 mg/kg of a levosimendan compound or a pharmaceutically acceptable salt thereof wherein the period between each intermittent dose is from 6 to 30 days. Even more specifically, the levosimendan compound or pharmaceutically acceptable salt thereof can be administered intermittently wherein the period between each intermittent dose is from 6 to 25 days.

Additionally, the dose of each intermittent intravenous infusion can be from 0.05 to 1 mg/kg of a levosimendan compound or a pharmaceutically acceptable salt thereof or from 0.1 to 0.6 mg/kg of a levosimendan compound or a pharmaceutically acceptable salt thereof.

Moreover, the intermittent intravenous infusion can be administered to a patient at a rate of 0.01-3 μg/kg/min of a levosimendan compound or a pharmaceutically acceptable salt thereof, at the rate of 0.02-1 μg/kg/min of a levosimendan compound or a pharmaceutically acceptable salt thereof or at the rate of 0.03-0.4 μg/kg/min of levosimendan or a pharmaceutically acceptable salt thereof.

The period between each intermittent dose can be determined by a variety of methods, such as, but not limited to, by echocardiography or by measuring the plasma level of natriuretic peptide.

In yet another embodiment, the present invention relates to a method of administering a levosimendan compound and a vasodilator to a patient. The method comprises the following steps: administering a first dose of a vasodilator or a levosimendan compound to a patient, followed by a rest period of at least one hour during which time said vasodilator or levosimendan compound is not administered and then administering at least a second dose of a vasodilator or a levosimendan compound. If the first dose comprises a vasodilator then the second dose comprises a levosimendan compound or if the first dose comprises a levosimendan compound then the second dose comprises a vasodilator. Additionally, the first or second dose delivers at least 0.0006 mg/kg of said levosimendan compound. The amount of vasodilator that can be administered to the patient is 0.00005 mg/kg to 15 mg/kg.

The period between each intermittent dose can be at least 2 hours, at least 4 hours, at least 6 hours, at least 12 hours, at least 24 hours, at least 30 hours, at least 2 days, at least 3 days, at least 4 days, at least 5 days, at least 6 days, at least 7 days, at least 8 days, at least 12 days, at least 13 days, at least 14 days, at least 21 days, at least 22 days, at least 25 days, at least 28 days, at least 30 days or at least 35 days.

The period between each intermittent dose can be determined by a variety of methods, such as, but not limited to, by echocardiography or by measuring the plasma level of natriuretic peptide.

The patient treated according to the above-described method may be suffering from pulmonary hypertension, myocardial ischemia, chronic heart failure, acute heart failure or acute on chronic heart failure.

The present invention also provides a kit that comprises:

a) a composition comprising a therapeutically effective amount of a levosimendan compound or a pharmaceutically acceptable salt thereof,

b) a package for containing said composition, and

c) instructions for administering intermittently doses of more than 0.04 mg/kg of the levosimendan compound or a pharmaceutically acceptable salt thereof to a patient, wherein the period between each intermittent dose is from 6 to 30 days, preferably from 6 to 25 days.

The method of the present invention provides a safe and effective short-term or long-term treatment of cardiovascular disorders, including but not limited to, acute heart failure, severe or less severe chronic heart failure and acute on chronic heart failure. The method can be used also in the treatment of other cardiovascular diseases such as myocardial ischemia or pulmonary hypertension.

### DETAILED DESCRIPTION OF THE INVENTION

The method of the invention relates to a method of administering an inotropic drug, such as a levosimendan compound, or a pharmaceutically acceptable salt thereof intermittently, i.e. by administering a plurality of intermittent doses, to a patient in need thereof. The intermittent administration according to the present invention is effective in reversing hemodynamic and neurohormonal disturbances in patients suffering from cardiovascular diseases, particularly patients suffering from pulmonary hypertension, myocardial ischemia, acute heart failure, chronic heart failure or acute on chronic heart failure. The intermittent administration can also be used prophylactically to prevent acute, chronic, acute on chronic, pulmonary hypertension and myocardial ischemia in a patient.

In particular, the intermittent administration according to the methods of the present invention provide important long-term benefits, such as increased cardiac output and decreased left ventricular filling pressure, with minimal risks in chronic heart failure patients. Levels of natriuretic peptides such as ANP (atrial natriuretic peptide) and BNP (brain natriuretic peptide) or their fragments (NT-proATP, NT-proBNP) which reflect myocardial pressure load, are also effectively suppressed. It was found that the plasma levels of these peptides were particularly suitable in monitoring the intermittent treatment of patients with chronic heart failure and in determining the suitable rest period between the intermittent doses.

The method of the invention also provides reduced risk of drug tolerance and improves patient compliance. In the intravenous setting, the method of the invention reduces the drawbacks associated with prolonged or continuous infusions such as weaning problems, increased risk of infection, long-term hospitalization and increased mortality.

The term “inotropic agent” refers to a drug that increases the force of myocardial contractility, with or without other physiological effects, such as, but not limited to, vasodilation, phosphodiesterase-inhibiting activity, etc. Inotropic agents are well known in the art and include, but are not limited to, levosimendan, dopamine, dobutamine, inamrinone, milrinone, dopexamine, digoxin, enoximone, pimobendan and metabolites thereof.

The term “intermittent” means administration that occurs non-continuously. Intermittent administration encompasses dosing of an established amount of the drug (such as an inotropic drug), with at least one rest period within the total administration period. Preferably, the rest period will be at least one hour but may be several hours, days, or weeks. For example, intermittent administration may occur once a week, once every second week, once every third week, and so on. The term intermittent also includes drug administration on consecutive days followed by period of days when no drug is administered. For example, the drug can be administered on day one and two and then again on day nine and ten, and so on. In a similar manner, the drug can be administered on day one and three. Thus, the intermittent dose can be administered over a period which ranges from minutes to several days. Furthermore, if an intermittent dose is 0.3 mg/kg, it can be dosed consecutively over three days at a daily dose of 0.1 mg/kg provided that there is at least one rest period during the 3 dosings. Alternatively, an intermittent dose of 0.3 mg/kg can be administered over three days as a first dose of 0.15 mg/kg, a second dose of 0.10 mg/kg and a third dose of 0.05 mg/kg. Many variations of intermittent administration will be apparent to those skilled in the art.

As used herein, the term “levosimendan compound” refers to any racemic mixture or enantiomer of levosimendan or a racemic mixture or enantiomer of the metabolites of levosimendan. The term “levosimendan” specifically refers to the (−)-enantiomer of [4-(1,4,5,6-tetrahydro-4-methyl-6-oxo-3-pyridazinyl)phenyl]hydrazono]propanedinitrile. The term also is intended to encompass combinations of levosimendan and its metabolites. A metabolite of levosimendan is, for example, (R)—N-[4-(1,4,5,6-tetrahydro-4-methyl-6-oxo-3-pyridazinyl)phenyl]acetamide.

The term “mg/kg of an inotropic agent or a pharmaceutically acceptable salt thereof” means milligram of an inotropic agent or a pharmaceutically acceptable salt thereof per one kilogram bodyweight of the patient, unless otherwise indicated. Similarly, the term “μg/kg of an inotropic agent or a pharmaceutically acceptable salt thereof” means microgram of an inotropic agent or a pharmaceutically acceptable salt thereof per one kilogram bodyweight of the patient.

The term “mg/kg of a levosimendan compound or a pharmaceutically acceptable salt thereof” means milligram of a levosimendan compound or a pharmaceutically acceptable salt thereof per one kilogram bodyweight of the patient, unless otherwise indicated. Similarly, the term “μg/kg of a levosimendan compound or a pharmaceutically acceptable salt thereof” means microgram of a levosimendan compound or a pharmaceutically acceptable salt thereof per one kilogram bodyweight of the patient.

The term “patient” means animals, preferably mammals, and humans.

As used herein, the term “acute heart failure” refers to a syndrome (namely, a collection of signs and symptoms) characterized by cardiac dysfunction caused by rapid deterioration of circulation dynamics. The onset of acute heart failure occurs over minutes to days. Common symptoms include, but are not limited to, dyspnea due to pulmonary congestion or cardiogenic shock due to low cardiac output, oliguria or anuria, cold extremities, hypotension, diaphoresis and dysrhythmias (tachyarrhythmias and bradydysrrhythmias), fatigue and/or confusion and/or altered mental status. Techniques and procedures for diagnosing a patient suffering from acute heart failure are well known to those skilled in the art.

As used herein, the term “chronic heart failure” refers to a progressive syndrome in which compensatory mechanisms deteriorate over time with progressive, unrelenting deterioration. Unlike acute heart failure, the onset of chronic heart failure occurs more slowly than over a period of minutes to days. Common symptoms include, but are not limited to, shortness of breath, fatigability, reduced exercise tolerance, while common signs include pulmonary congestion and crackles, enlargement of the liver and spleen, pitting edema, gallop rhythms, and elevated jugular venous pressures. Techniques and procedures for diagnosing a patient suffering from chronic heart failure are well known to those skilled in the art.

As used herein, the term “acute on chronic heart failure” refers to a syndrome characterized by cardiac dysfunction caused by rapid deterioration of circulatory dynamics in a patient with a history of heart failure. Common symptoms include, but are not limited to, edema, dyspnea due to pulmonary congestion or cardiogenic shock due to low cardiac output, oliguria or anuria, cold extremities, hypotension, diaphoresis, and dysrhythmias (tachyarrhythmias and bradydysrrhythmias), fatigue and/or confusion and/or altered mental status. Techniques and procedures for diagnosing a patient suffering from acute on chronic heart failure are well known to those skilled in the art.

The terms “treating”, “treat” or “treatment” includes preventive (e.g. prophylactic) and palliative treatment.

As used herein, the phrase “pharmaceutically acceptable” refers to a form of an ingredient that is physiologically suitable for pharmaceutical use. For example, the phrase “pharmaceutically acceptable salt” refers to the salt forms of an active ingredient, such as an inotropic agent (including, but not limited to, a levosimendan compound or a pharmaceutically acceptable salt thereof), that is physiologically suitable for pharmaceutical use.

As used herein, the term “vasodilator(s)” refers to a drug that opens the arteries and veins thereby reducing the heart's workload and allowing more blood to reach the tissues. Several types of vasodilators are known in the art and include, but are not limited to, hydralizine, hydralazine hydrochloride, nicorandil, fenoldopam, natriuretic peptides, natrecor, nesiritide, nitroprusside, nitroprusside sodium, nipride, milrinone, primacor, nitroglycerin, glyceryl trinitrate, isosorbide dinitrate, isosorbide mononitrate and metabolites thereof.

The term “mg/kg of a vasodilator” means milligram of a vasodilator of per one kilogram bodyweight of the patient, unless otherwise indicated. Similarly, the term “μg/kg of a vasodilator” means microgram of a vasodilator hereof per one kilogram bodyweight of the patient.

The strength of each intermittent dose to be administered to a patient depends e.g. upon the condition to be treated, the method of administration, age and the condition of the patient. In general, each intermittent dose comprises at least 0.0006 mg/kg of an inotropic drug or a pharmaceutically acceptable salt thereof. For example, in one embodiment, the present invention contemplates a method wherein a first dose of an inotropic agent or a pharmaceutically acceptable salt thereof other than dobutamine is administered to a patient. After the first dose, there is a rest period (an initial rest period), during which the patient is not administered any inotropic agent. Preferably, this rest period is at least one hour. After this rest period, a second dose of an inotropic agent or a pharmaceutically acceptable salt thereof is administered to the patient. In the above-described method, at least one of the inotropic agents used in the first dose or the second dose is a levosimendan compound or a pharmaceutically acceptable salt thereof. Additionally, in the above described method, each of the first and second dose of inotropic agent delivers at least 0.0006 mg/kg of said inotropic agent to the patient.

Moreover, in the above described method, after the patient is administered a second dose of the inotropic agent, the patient can be subjected to a further rest period of at least one hour (a subsequent rest period). After this subsequent rest period, the patient can be administered another dose of an inotropic agent, namely, a third dose can be given. This dose must deliver at least 0.0006 mg/kg of said inotropic agent to the patient. These subsequent rest periods and further dosings with an inotropic agent can be repeated for as long as necessary to adequately treat the patient.

In yet another embodiment, the present invention contemplates a method wherein a levosimendan compound and a vasodilator are administered to a patient. More specifically, a first dose of a vasodilator or a levosimendan compound is administered to a patient. After the first dose, there is a rest period (an initial rest period), during which the patient is not administered any vasodilator or levosimendan compound. Preferably, this rest period is at least one hour. After this rest period, a second dose of a vasodilator or a levosimendan compound is administered to the patient. In the above-described method, if a vasodilator is administered to a patient as the first dose then a levosimendan compound is administered to the patient in the second dose. Alternatively, if the first dose is a levosimendan compound then the second dose is a vasodilator. Additionally, in the above described method, at least 0.0006 mg/kg of said levosimendan compound must be delivered to a patient.

Moreover, in the above described method, after the patient is administered a second dose of either a vasodilator or a levosimendan compound, the patient can be subjected to a further rest period of at least one hour (a subsequent rest period). After this subsequent rest period, the patient can be administered another dose of a vasodilator or a levosimendan compound as necessary to adequately treat the patient. If a levosimendan compound is administered than the dose must deliver least 0.0006 mg/kg of said levosimendan compound to the patient. These subsequent rest periods and further dosings with a vasodilator or a levosimendan compound can be repeated for as long as necessary to adequately treat the patient.

The vasodilator can be administered intravenously to a patient in the above described method in the amount of 0.00005 mg/kg to 15 mg/kg. For example, the vasodilator can be administered to a patient in the amount of 0.00005 to 0.300 mg/kg, in the amount of 0.05 to 0.300 mg/kg or in the amount of 0.25 to 15.0 mg/kg. Additionally, the vasodilator can be administered at a rate of 0.0001-15 μg/kg/min (also referred to herein as “mcg/kg/min”). For example, a vasodilator such as can be administered at a rate of 0.001-0.10 μg/kg/min, at a rate of 0.05-3.00 μg/kg/min or at a rate of 0.25-10.0 μg/kg/min. If an initial intravenous bolus is needed, an intravenous bolus of 0.010-50 μg/kg of a vasodilator followed by maintenance infusion at the rate as described above can be used. For example, the initial loading dose can be 0.010-0.725 μg/kg or 1-2 μg/kg.

In another embodiment, the present invention also contemplates a method of administering a levosimendan compound or a pharmaceutically acceptable salt thereof to a patient by administering intermittently a dose of at least 0.0006 mg/kg of a levosimendan compound or a pharmaceutically acceptable salt thereof to a patient where the period between each intermittent dose is at least one hour. As mentioned previously, the intermittent dose is at least 0.0006 mg/kg. Additionally, the present invention also contemplates that each intermittent dose can contain more than 0.04 mg/kg of a levosimendan compound or a pharmaceutically acceptable salt thereof. Moreover, each intermittent dose can comprise from about 0.05 to about 1 mg/kg, for example, from about 0.1 to about 0.6 mg/kg, of a levosimendan compound or a pharmaceutically acceptable salt thereof, depending on the period over which the intermittent dose is administered.

Typically, in the methods of the present invention, each intermittent dose is administered over a period which ranges from minutes, to several hours, to several days, to several weeks. Suitably, each intermittent dose is administered over the period, which is less than 7 days, less than 5 days, less than 3 days, less than 2 days and less than 24 hours.

As mentioned previously herein, the period between each intermittent dose (namely, the rest period during which the inotropic agent (such as, but not limited to, a levosimendan compound or a pharmaceutically acceptable salt thereof) or the vasodilator) is not administered is at least one hour. The present invention further contemplates that the period between each intermittent dose is at least 2 hours, at least 4 hours, at least 6 hours, at least 12 hours, at least 24 hours, at least 30 hours, at least 2 days, at least 3 days, at least 4 days, at least 5 days, at least 6 days, at least 7 days, at least 8 days, at least 12 days, at least 13 days, at least 14 days, at least 21 days, at least 22 days, at least 25 days, at least 28 days, at least 30 days or at least 35 days. Moreover, the present invention also contemplates that the period between each intermittent dose can be from about 6 to about 30 days, 6 to 25 days, more preferably from 7 to 22 days, from about 7 to about 16 days, or from 8 to 13 days.

The administration of the intermittent dose can be by e.g. parenteral, oral, transmucosal or transdermal route.

In general, an inotropic agent, such as a levosimendan compound, can be administered orally to man in a daily dose ranging from about 0.01 to about 0.2 mg/kg, more typically from 0.02 to 0.15 mg/kg, depending on the age, body weight and condition of the patient.

According to one preferred embodiment of the invention the administration of the intermittent dose is by intravenous route, suitably by intravenous infusion.

According to one particularly preferred embodiment of the invention the intermittent dose (inotropic agent (such as a levosimendan compound) or vasodilator) is given by an intravenous infusion over the period of 1 hour-2 days (48 hours), preferably over the period of 4-36 hours, more preferably over the period of 6-30 hours; for example by an intravenous infusion lasting 24 hours, 12 hours, 8 hours or 6 hours. In another preferred embodiment of the invention, the intermittent dose is given by an intravenous infusion of less than 24 hours.

Suitably, the intermittent intravenous infusion can be administered at the rate of 0.01-3 μg/kg/min (also referred to herein as “mcg/kg/min”), more preferably at the rate of 0.02-1 μg/kg/min, still more preferably at the rate of 0.03-0.4 μg/kg/min, of an inotropic agent (such as levosimendan compound or a pharmaceutically acceptable salt thereof). If an initial intravenous bolus is needed, an intravenous bolus of 1-100 μg/kg, preferably 5-50 μg/kg, of an inotropic agent, such as, a levosimendan compound or a pharmaceutically acceptable salt thereof, followed by maintenance infusion at the rate as described above can be used.

Intermittent continuous infusion at the rate of 0.1-0.2 μg/kg/min, optionally with a initial loading dose of 10-15 μg/kg, of a levosimendan compound or a pharmaceutically acceptable salt thereof for 24 hours followed by a rest period of 7 to 22 days, preferably from 7 to 16 days, more preferably from 8 to 13 days, has been found to be particularly suitable for the treatment of chronic heart failure, particularly severe chronic heart failure.

If desired, the length of the rest period (i.e. the period when no inotropic agent or vasodilator is administered), can be determined in each patient using suitable invasive or non-invasive monitoring means, non-invasive means being preferred. Such invasive and non-invasive means are well known to those of ordinary skill in the art. A particularly suitable non-invasive means is echocardiography. Furthermore, natriuretic peptides ANP (atrial natriuretic peptide), BNP (brain natriuretic peptide) and/or or their fragments (NT-proATP, NT-proBNP) are particularly suitable as markers and may be used to determine the length of the rest period between intermittent infusions. Other methods that can be used include monitoring weight changes in a patient, patient symptoms or by quality of life metrics.

An inotropic agent, such as a levosimendan compound or a pharmaceutically acceptable salt thereof is formulated into dosage forms using principles well known to practitioners in the art. It is given to a patient as such or preferably in combination with suitable pharmaceutical excipients in the form of tablets, granules, capsules, suppositories, emulsions, suspensions or solutions whereby the contents of the active compound in the formulation is from about 0.1 to 100% per weight. Choosing suitable ingredients for the composition is routine for those of ordinary skill in the art. It is evident that suitable carriers, solvents, gel forming ingredients, dispersion forming ingredients, antioxidants, colours, sweeteners, wetting compounds, release controlling components and other ingredients normally used in this field of technology also may be used.

For oral administration in tablet or capsule form, suitable carriers and excipients include e.g. lactose, corn starch, magnesium stearate, calcium phosphate and talc. For controlled release oral compositions release controlling components can be used. Typical release controlling components include hydrophilic gel forming polymers such as hydroxypropylmethyl cellulose, hydroxypropyl cellulose, carboxymethyl celluloses, alginic acid or a mixture thereof; vegetable fats and oils including vegetable solid oils such as hydrogenated soybean oil, hardened castor oil or castor seed oil.

Tablets can be prepared by mixing the active ingredient with the carriers and excipients and compressing the powdery mixture into tablets. Capsules can be prepared by mixing the active ingredient with the carriers and excipients and placing the powdery mixture in capsules, e.g. hard gelatin capsules. Typically a tablet or a capsule comprises from about 0.1 to 10 mg, more typically 0.2 to 5 mg, of levosimendan or a pharmaceutically acceptable salt thereof.

Formulations suitable for intravenous administration such as injection or infusion formulation comprise sterile isotonic solutions of a levosimendan compound or a pharmaceutically acceptable salt thereof and vehicle, preferably pharmaceutically acceptable aqueous solutions. Typically an intravenous infusion solution comprises from about 0.001 to 1, preferably from about 0.01 to 0.1 mg/ml, of a levosimendan compound or a pharmaceutically acceptable salt thereof. The formulation for intravenous administration may also be in the form of an infusion concentrate, which is diluted with an aqueous vehicle before use. Typically such infusion concentrate comprises a levosimendan compound or a pharmaceutically acceptable salt thereof dissolved in dehydrated ethanol. A preferred formulation is described in WO 01/19334, published Mar. 22, 2001.

The present invention also provides a kit comprising

a) a composition comprising a therapeutically effective amount of an inotrophic compound or a pharmaceutically acceptable salt thereof,

b) a package for containing said composition, and

c) instructions for administering intermittently doses of more than 0.0006 mg/kg of the inotropic compound or a pharmaceutically acceptable salt thereof to a patient, wherein the period between each intermittent dose is at least one hour.

The composition of the above kit comprising a therapeutically effective amount of a levosimendan compound or a pharmaceutically acceptable salt thereof may be any of the formulations described above, e.g. in the form of tablets, granules, capsules, suppositories, emulsions, suspensions or solutions whereby the contents of the active compound in the formulation is from about 0.1 to 100% per weight. The package may be in any form normally used in the art, e.g. a bottle, blister, syringe, bag, box, and the like, depending on the nature of the composition. Typically, the kit comprises instructions for the intermittent administration of the composition in accordance to the method of the present invention.

Salts of levosimendan may be prepared by known methods. Pharmaceutically acceptable salts are useful as active medicaments, however, preferred salts are the salts with alkali or alkaline earth metals.

By way of example and not of limitation, examples of the present invention will now be given.

### EXAMPLES

### Example 1

### Pharmaceutical Examples

### Example 1a

### Oral Capsule

Hard gelatin capsule size 3

| | | |
|---|---|---|
| Levosimendan | 2.0 | mg |
| Lactose | 198 | mg |

The pharmaceutical preparation in the form of a capsule was prepared by mixing levosimendan with lactose and placing the powdery mixture in bard gelatin capsule.

### Example 1b

### Concentrate Solution for Intravenous Infusion

| | | |
|---|---|---|
| (a) levosimendan | 2.5 | mg/ml |
| (b) Kollidon PF12 | 10 | mg/ml |
| (c) citric acid | 2 | mg/ml |

| | |
|---|---|
| (d) dehydrated ethanol | ad 1 ml (785 mg) |

The concentrate solution was prepared by dissolving citric acid, Kollidon PF121 and levosimendan to dehydrated ethanol in the sterilized preparation vessel under stirring. The resulting bulk solution was filtered through a sterile filter (0.22 μm). The sterile filtered bulk solution was then aseptically filled into 8 ml and 10 ml injection vials (with 5 ml and 10 ml filling volumes) and closed with rubber closures.

The concentrate solution for intravenous infusion is diluted with an aqueous vehicle before use. Typically the concentrate solution is diluted with aqueous isotonic vehicles, such as 5% glucose solution or 0.9% NaCl solution so as to obtain an aqueous intravenous solution, wherein the amount of levosimendan is generally within the range of about 0.001-1.0 mg/ml, preferably about 0.01-0.1 mg/ml.

### Experiments

### Example 1

Methods

A double-blind, placebo-controlled, parallel group, single center study in patients with New York Heart Association functional class II to III heart failure was conducted. Patients were randomised in 1:1 ratio to receive either levosimendan or placebo. Study drug administration was initiated with a loading dose of 12 μg/kg of levosimendan or placebo delivered over 10 minutes. This was followed by a continuous infusion of 0.1 μg/kg/min for 50 minutes. If the dose was well tolerated the infusion rate was increased to 0.2 μg/kg/min for a further 23 hours. After the study drug was started, the hemodynamic assessments were repeated at 30 minutes, at two and six hours. The non-invasive hemodynamic assessments were repeated at 24-hours. Follow-up visits took place at 2, 3, 5, 7, 9 and 14 days from the beginning of the study. On these visits echocardiographic measurements were performed and blood pressure and heart rate were measured.

An Acuson Sequoia ultrasound system with 2.5-3.75 MHz probes was used for the Doppler echocardiographic measurements. Blood flow velocity curves were recorded at a sweep speed of 100 mm/s. Left ventricular ejection fraction was assessed by two-dimensional apical two- and four-chamber views with use of modified Simpson rule. Mitral flow velocity was assessed by pulsed wave Doppler from the apical four-chamber view by placing a 3-mm sample volume between the tip of the mitral leaflets in diastole. The following measurements were made over five consecutive cycles: maximal early and maximal late diastolic velocity and their ratio, duration of the late diastolic velocity wave, deceleration time and deceleration rate of early diastolic velocity. Peak velocities were defined as the highest point of the spectrum.

Blood samples (4 ml) for natriuretic peptide (NT-proANP, NT-proBNP) measurements were taken into pre-cooled EDTA tubes. Samples were taken at baseline (0-sample) and on the first study day 30 min, 2 h, 6 h and 24 h after the start of the infusion and in the mornings of days 2, 3, 5, 7, 9 and 14 after the start of the study drug infusion.

Results

Invasively measured cardiac output (CO) increased from 4.3 L/min to 5.4 L/min in levosimendan group at six hours. PCWP decreased from 20 mmHg to 15 mmHg in response to the levosimendan treatment, whereas a small increase in PCWP (17 mmHg-20 mmHg) was observed in placebo group.

Echocardiographically estimated PCWP reached its lowest value 2 days after starting the infusion, whereas the highest CO value estimated by echocardiography was detected at the end of the 24-hour infusion. The linearly estimated duration of the decrease in PCWP was 9 days, and the duration of an increase in CO was 13 days. Plasma NT-proANP and NT-proBNP levels reached their lowest values at days 3 and 2, and by a linear model the treatment effect was estimated to last 16 and 12 days, respectively. NT-proANP and NT-proBNP levels closely coincided with the sustained hemodynamic response in the patients.

Conclusions: A 24-hour levosimendan infusion achieved a rapid improvement in the hemodynamic parameters of patients with NYHA II-III congestive heart failure with maximal effects occurring during 1-3 days after starting the infusion. The beneficial hemodynamic and neurohormonal effects were maintained up to two weeks after levosimendan administration. It is concluded that a 24-hour levosimendan infusion of 0.2 μg/kg/min given intermittently every 10 days is effective in achieving clinical benefits with minimal risks in patients suffering from severe chronic heart failure.

### Example 2

### Potential Chronic Intermittent Therapy Study

This example describes a proposed study that can be performed by those skilled in the art to examine the survival of patients with chronic heart failure when said patients are administered intravenous levosimendan versus intravenous placebo or some other comparator drug on an intermittent basis.

The rationale for this study would be that intermittent doses of levosimendan will provide a benefit to patients with chronic heart failure. This benefit will include, but would not be limited to, a prevention or reduction in hospitalizations for acute heart failure with improvement in clinical status (such as, but not limited to, fatigue, quality of life, dyspnea, etc.)

Population: Patients with a history of chronic heart failure that have been hospitalized at least once in the last six months for acute heart failure. These patients will have an ejection fraction of less than or equal to 35%.

Duration: Patients will be dosed for at least 3 months and followed for a total of six months.

Inclusion criteria (Meeting all of the following below):

History of CHF.
Hospitalization for Acute Heart Failure in last 6 months.
Ejection Fraction less than or equal to 35% in last six months.
Ongoing therapy with ACE-I or ARBs, Beta-blockers, aldosterone antagonists, digoxin, and oral loop diuretics (the dose should be stable for 1 month prior to study entry).

Other inclusion criteria could be added if deemed to be necessary.

Exclusion criteria (Meeting any one of the following below):

History of Torsade de Pointes.
Creatinine clearance less than 30 ml/min.
History of ventricular tachycardia or ventricular fibrillation and no implantable cardioverter defibrillator (“ICD”).
Baseline heart rate greater than 100 bpm.
Baseline Systolic blood pressure less than 90 mm Hg.

Other exclusion criteria could be added if deemed to be necessary.

Study Procedures:

Vitals signs (such as blood pressure, heart rate, etc.) will be collected prior to the infusion and every 30 minutes during the infusion, and one hour after the infusion.
Electrolytes, BUN, creatinine, BNP, plasma renin activity, serum aldosterone levels will be collected before study drug infusion and one hour after.
ECGs will be collected before study drug infusion and one hour after.

Other procedures could be added if needed.
Dosing Regimen: There will be multiple arms including a placebo or other comparator arm.
Dose (levosimendan or placebo or comparator) will range from 0.01 mcg/kg/min and higher (such as from 0.01 mcg/kg/min to 3 mcg/kg/min). The dosing for the first administration and the re-administration(s) may be the same or they may be different.
Infusion will be given for at least 1 hour.
The patient will be required to stay in the infusion center for at least 2 hours following the completion of the infusion.
There will be differing intervals between infusions. (or re-administrations), such as 2 weeks and 4 weeks.

If the levosimendan or placebo or other comparator is not well tolerated, then it will not be re-administered.

Administration: Levosimendan can be supplied as a concentrated solution (2.5 mg/mL). It can be administered as an intravenous infusion (for example as 50 μg/mL in 5% glucose) through any means known in the art (such as a central or peripheral line).

Primary Endpoint:

A composite of death and hospitalizations (including emergency room visits, and urgent clinic visits) over the 6-month study period will be compared between treatment groups (levosimendan and placebo).

Secondary Endpoints can include the following:

Number of days alive out of the hospital (“DAOH”) over 6 months.
Change in quality of life over the 6 month period.
Mean plasma BNP over the 6 month period.
All-cause mortality over the 6 month period.
Change in estimated glomerular filtration rate (“eGFR”) from baseline to 6 months.
Change in weight from baseline to 6 months.
   Location: This will be a randomized, double-blinded multi-center study, multi-national study.

### Example 3

### Proposed Intermittent Dosing Study

This example describes a proposed study that can be performed by those skilled in the art to examine the survival of patients with chronic heart failure when said patients are administered intravenous levosimendan versus intravenous placebo or some other comparator on an intermittent basis.

The rationale for this study would be:

With the long half life of the levosimendan metabolite, there is a beneficial effect of levosimendan lasting ≧2 weeks.

Repeated intravenous dosing at every 1 to 4 weeks for at least 3 doses will give continuous supportive therapy.

Myocardial function can be improved.

Improve patient function.

Reverse or prevent myocardial remodeling.

Prolong survival.

Decrease hospitalizations.

Unmet Medical Need:

Class IIIb/IV patients have little option short of cardiac transplant to improve function and quality of life (QOL).

Intermittent infusion of dobutamine or milrinone improves symptoms for a short time, but does not positively impact longer term QOL, morbidity or mortality.

There is a high pharmacoeconomic cost to current management of these patients.

Design:

This study would be a randomized, blinded study of patients comparing intravenous levosimendan to placebo or other comparator, both in addition to standard of care therapy on days alive and out of hospital at 180 day follow-up.

Primary Endpoint:

Days alive and out of hospital at 180 days following required repeated doses of study drug.

Secondary Endpoints:

All cause mortality at 180 days.

Time to first or re-hospitalization following study drug administration.

Requirement for rescue intravenous medication.

Number of cardiac events requiring intervention during 180 day follow-up.

Renal function measurements.

BNP determinations.

Echocardiography at baseline and at follow-up.

Functional assessment, QOL determination.

Drug Administration:

1^{st}—24 hour continuous infusion of study drug without loading dose, such as 0.1 μg/kg/min levosimendan or equivalent volume placebo or other comparator; with titration up to 0.2 μg/kg/min for effect or down to 0.05 μg/kg/min for based on tolerability and/or patient response.

Subsequent doses at every 1 to 4 weeks, 2-8 hour infusion of levosimendan or equal volume placebo or other comparator at dose tolerated during previous administration, titrated to effect as needed following forced repetition out to 180 days.

Levosimendan can be supplied as a concentrated solution (2.5 mg/mL). It can be administered as an intravenous infusion (for example, 50 μg/mL in 5% glucose) through any means known in the art (such as a central or peripheral line).

Patient Population:

Class III/IV patients admitted for worsening heart failure who are symptomatic despite optimal oral drug therapy and life style adjustments i.e., diet. 500 patients at ˜40 sites worldwide which have an established ability to follow the study.

Assumptions:

Standard of care in this population will result in 40% cardiovascular (“CV”) event rate at 180 days with overall 30% mortality.

Event rate and mortality will be decreased by at least 10% by levosimendan.

Labeling:

Repeated administrations of levosimendan.

Improved survival at 6 months.

Pharmacoeconomic benefit including quality of life.

Decreased number of hospitalizations and the length of said hospitalizations.

1. A method of administering a levosimendan or a pharmaceutically acceptable salt thereof to a patient, the method comprising the step of administering intermittently a dose of more than 0.04 mg/kg of a levosimendan or a pharmaceutically acceptable salt thereof to a patient, wherein the period between each intermittent dose is from 6 to 25 days.

2. A method according to claim 1, wherein the period between each intermittent dose is from 7 to 22 days.

3. (canceled)

4. A method according to claim 1, wherein the patient suffers from chronic heart failure.

5. A method according to claim 1, wherein the patient suffers from acute heart failure.

6. A method according to claim 1, wherein the patient suffers from acute on chronic heart failure.

7. A method of claim 1, wherein the patient suffers from pulmonary hypertension.

8. A method according to claim 1, which comprises performing an echocardiography on the patient to select the period between each intermittent dose.

9. A method according to claim 1, which comprises measuring the patient's plasma level of a natriuretic peptide or a fragment thereof to select the period between each intermittent dose.

10. A method of administering a levosimendan or a pharmaceutically acceptable salt thereof to a patient suffering from chronic heart failure, the method comprising the step of administering intermittently a dose of more than 0.04 mg/kg of a levosimendan or a pharmaceutically acceptable salt thereof to a patient, wherein the period between each intermittent dose is from 6 to 25 days.

11. A method according to claim 10, wherein the period between each intermittent dose is from 7 to 22 days.

12. A method of administering an inotropic agent or a pharmaceutically acceptable salt thereof to a patient, the method comprising the steps of: administering a first dose of an inotropic agent or a pharmaceutically acceptable salt thereof other than dobutamine to a patient and then administering at least a second dose of an inotropic agent or a pharmaceutically acceptable salt thereof to the patient, wherein each said dose delivers at least 0.0006 mg/kg of said inotropic agent or a pharmaceutically acceptable salt thereof, wherein at least one of the inotropic agents used in the first dose or the second dose is a levosimendan compound or a pharmaceutically acceptable salt thereof, and further wherein the period between the first dose and the second dose during which time the patient is not administered any inotropic agent or a pharmaceutically acceptable salt thereof is from 8 to 25 days.

13. A method according to claim 12, wherein the period between each intermittent dose is selected from the group consisting of: at least 12 days, at least 13 days, at least 14 days, at least 21 days, and at least 22 days.

14. (canceled)

15. A method according to claim 12, wherein the patient suffers from chronic heart failure.

16. A method according to claim 12, wherein the patient suffers from acute heart failure.

17. A method according to claim 12, wherein the patient suffers from acute on chronic heart failure.

18. A method according to claim 12, wherein the patient suffers from pulmonary hypertension.

19. A method according to claim 12, which comprises performing an echocardiography on the patient to select the period between the first dose and the second dose.

20. A method according to claim 12, which comprises measuring the patient's plasma level of a natriuretic peptide or a fragment thereof to select the period between the first dose and the second dose.

21. A method of administering a levosimendan or a pharmaceutically acceptable salt thereof to a patient, the method comprising the step of: administering to a patient intermittent intravenous infusions, each infusion delivering a dose of more than 0.04 mg/kg of a levosimendan or a pharmaceutically acceptable salt thereof to the patient, and wherein the period between each intermittent infusion is from 6 to 25 days.

22. A method according to claim 21, wherein the period between each intermittent dose is selected from the group consisting of: at least 7 days, at least 8 days, at least 12 days, at least 13 days, at least 14 days, at least 21 days, and at least 22 days.

23-24. (canceled)

25. A method according to claim 21, wherein the dose of each intermittent intravenous infusion is from 0.05 to 1 mg/kg of a levosimendan or a pharmaceutically acceptable salt thereof.

26. A method according to claim 25, wherein the dose of each intermittent intravenous infusion is 0.1 to 0.6 mg/kg of a levosimendan or a pharmaceutically acceptable salt thereof.

27. A method according to claim 21, wherein the administration of each intermittent intravenous infusion is over the period of 1 hour to 2 days.

28. A method according to claim 27, wherein the administration of each intermittent intravenous infusion is over the period of 4-36 hours.

29. A method according to claim 28, wherein the administration of each intermittent intravenous infusion is over the period of about 6-30 hours.

30. A method according to claim 21, wherein the administration of each intermittent intravenous infusion is over a period of less than 24 hours.

31. A method according to claim 21, wherein the period between each intermittent dose is from 7 to 22 days.

32. A method according to claim 31, wherein the period between each intermittent dose is from 7 to 16 days.

33. A method according to claim 32, wherein the period between each intermittent dose is from 8 to 13 days.

34. A method according to claim 21, wherein the intermittent intravenous infusion is administered at the rate of 0.01-3 μg/kg/min of a levosimendan or a pharmaceutically acceptable salt thereof.

35. A method according to claim 34, wherein the intermittent intravenous infusion is administered at the rate of 0.02-1 μg/kg/min of a levosimendan or a pharmaceutically acceptable salt thereof.

36. A method according to claim 35, wherein the intermittent intravenous infusion is administered at the rate of 0.03-0.4 μg/kg/min of levosimendan or a pharmaceutically acceptable salt thereof.

37. A method according to claim 21, which comprises performing an echocardiography on the patient to select the period between each intermittent dose.

38. A method according to claim 21, which comprises measuring the patient's plasma level of a natriuretic peptide or a fragment thereof to select the period between each intermittent dose.

39. A method of administering a levosimendan or a pharmaceutically acceptable salt thereof and a vasodilator to a patient, the method comprising the steps of: administering a first dose of a vasodilator or levosimendan or a pharmaceutically acceptable salt thereof to a patient and then administering at least a second dose of a vasodilator or a levosimendan or a pharmaceutically acceptable salt thereof, wherein if the first dose comprises a vasodilator then the second dose comprises a levosimendan or a pharmaceutically acceptable salt thereof or if the first dose comprises a levosimendan or a pharmaceutically acceptable salt thereof then the second dose comprises a vasodilator, and further wherein the first or second dose delivers at least 0.0006 mg/kg of levosimendan or a pharmaceutically acceptable salt thereof and even further wherein the period between the first dose and the second dose during which time said vasodilator or levosimendan or a pharmaceutically acceptable salt thereof is not administered is at least 5 days.

40. A method according to claim 39, wherein the period between the intermittent dose is selected from the group consisting of: at least 6 days, at least 7 days, at least 8 days, at least 12 days, at least 13 days, at least 14 days, at least 21 days, at least 22 days, at least 25 days, at least 28 days, at least 30 days and at least 35 days.

41. A method according to claim 39, wherein the patient is administered 0.00005 mg/kg to 15 mg/kg of a vasodilator.

42. A method according to claim 39, wherein the patient suffers from chronic heart failure.

43. A method according to claim 39, wherein the patient suffers from acute heart failure.

44. A method according to claim 39, wherein the patient suffers from acute on chronic heart failure.

45. A method according to claim 39, wherein the patient suffers from pulmonary hypertension.

46. A method according to claim 39, which comprises performing an echocardiography on the patient to select the period between the first dose and the second dose.

47. A method according to claim 39, which comprises measuring the patient's plasma level of a natriuretic peptide or a fragment thereof to select the period between the first dose and the second dose.

48. A kit that comprises:
a) a composition comprising a therapeutically effective amount of a levosimendan or a pharmaceutically acceptable salt thereof,
b) a package for containing said composition, and
c) instructions for administering intermittently doses of more than 0.04 mg/kg of a levosimendan or a pharmaceutically acceptable salt thereof to a patient, wherein the period between each intermittent dose is from 6 to 25 days.

49. (canceled)

50. A method according to claim 39, wherein the vasodilator is selected from the group consisting of: hydralizine, hydralazine hydrochloride, nicorandil, fenoldopam, natriuretic peptides, natrecor, nesiritide, nitroprusside, nitroprusside sodium, nipride, milrinone, primacor, nitroglycerin, glyceryl trinitrate, isosorbide dinitrate, isosorbide mononitrate and metabolites thereof.
